(19) United States
(12) Patent Application Publication
(10) Pub. No.: US 2026/0131010 A1
(43) Pub. Date: May 14, 2026

(54) MENIN PROTEOLYSIS TARGETING CHIMERAS (PROTACS)

(71) Applicant: Board of Regents, The University of Texas System, Austin, TX (US)

(72) Inventors: Karinel Nieves-Merced, San Antonio, TX (US); Daifeng Jiang, San Antonio, TX (US); Matthew J. Hart, San Antonio, TX (US); Stanton F. McHardy, San Antonio, TX (US)

(21) Appl. No.: 18/862,886

(22) PCT Filed: May 3, 2023
(86) PCT No.: PCT/US2023/020789

§ 371 Date: Nov. 4, 2024

Related U.S. Application Data
(60) Provisional application No. 63/337,730, filed on May 3, 2022.

(51) Int. Cl. A61K 47/55

(52) U.S. Cl. CPC A61K 47/55

(57) ABSTRACT

Described herein are Proteolysis targeting chimeras (PROTACs) compounds, compositions, and methods for the treatment of various cancers. In particular, Menin protein targeting PROTACs are described.

### RELATED APPLICATIONS

This Application claims priority to U.S. Provisional Application 63/337,730 filed May 3, 2022 which is incorporated herein by reference in its entirety.

### STATEMENT REGARDING FEDERALLY FUNDED RESEARCH

This invention was made with government support under 5UL1TR002645-04 awarded by the National Institutes of Health. The government has certain rights in the invention.

### BACKGROUND

Mixed lineage leukemia (MLL) is a very aggressive blood cancer that predominantly occurs in pediatric patients and is characterized by the presence of MLL fusion proteins that are the result of chromosomal translocations affecting the MLL gene at 11q23.1 A critical component of MLL-fusion protein complex is a protein called Menin. Numerous studies have demonstrated a critical role of Menin as an oncogenic cofactor in leukemic transformations mediated by MLL fusion proteins. MI-503 is a small molecule inhibitor that binds to Menin and inhibits its interaction with MLL-fusion proteins. MI-503 has been shown to be effective in mouse models of MLL leukemias (Borkin, et al. Cancer Cell 2015, 27(4) 589-602). Proteolysis targeting chimeras (PROTACs) are compounds that target proteins for degradation by hijacking the activity of E3 ubiquitin ligases to promote degradation. PROTACs are particularly effective in cancers of the circulatory system and have the advantage over traditional therapeutics in that permanent occupancy of the targeted protein is not necessary as the PROTAC uses the in vivo degradation machinery to catalytically degrade proteins of interest.

There remains a need for additional compositions and methods for the treatment of cancers and particularly additional PROTACs, particularly targeting Menin.

### SUMMARY

Embodiments described herein provide additional compositions and methods for the treatment of various cancers. In particular, compounds directed to Menin PROTACs are described as a solution to current problems associated with the treatment of cancer.

Certain embodiments are directed to compounds or PROTACs having the general structure of M-L-E, E-L-M, or a pharmaceutically acceptable salt thereof, wherein M is a Menin protein binding moiety; E is an E3 ubiquitin ligase protein binding ligand; and L is a linker group.

Compounds or PROTACs of the invention can have a structure of Formula I or Formula II:

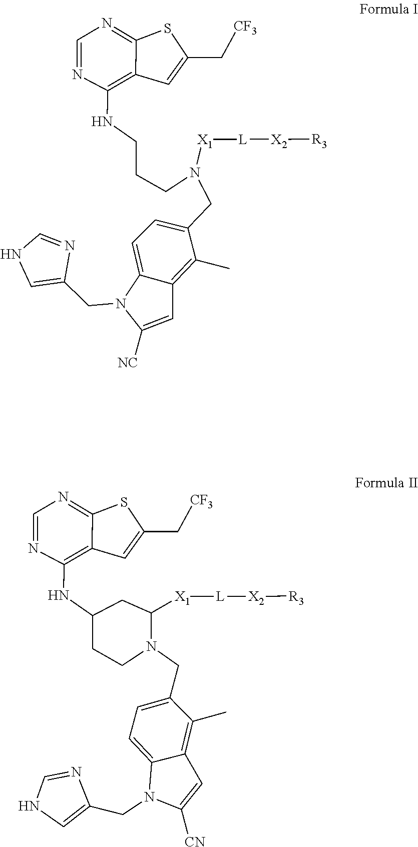

wherein spacers X₁ and X₂ are independently selected from an optionally substituted methylene, nitrogen or oxygen, including —NH—C(O)—, —C(O)—NH—, —C(O)—O—, —O—C(O)—, —O—CH₂—, —NH—CH₂—, —CH₂—NH—, —CH₂—O—, —C(O)—NH—CH₂-triazole-CH₂—, —NH—C(O)—CH₂-triazole-CH₂—, —C(O)—CH₂—, —CH₂—C(O)—, —C(O)—NH—CH₂—, or —CH₂—NH—C(O)—; L is a linker; and R₃ is an E3 binding moiety.

The linker (L) can be selected from alkyl chains (C1-C12), branched alkyl chains (C1-C10), repeating ethylene glycol chains, i.e. (—O—CH₂-CH₂—O—)ₙ, where n is 1-8, ethylene diamines i.e. (—NH—CH₂-CH₂—NH—)ₙ, where n is 1-8, and substituted cyclic amines. The substituted cyclic amine can have a structure of

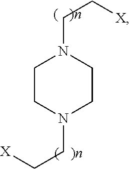

wherein n=1-6.

The E3 binding moiety can be selected from a moiety that binds Von Hippel-Lindau (VHL) E3 ubiquitin ligase, moiety that binds cereblon (CRBN) E3 ubiquitin ligase, moiety that binds inhibitor of apoptosis protein (IAP) E3 ubiquitin ligase, or moiety that binds mouse double minute 2 homolog (MDM2) E3 ubiquitin ligase. In certain aspects the E3 binding moiety is selected from:

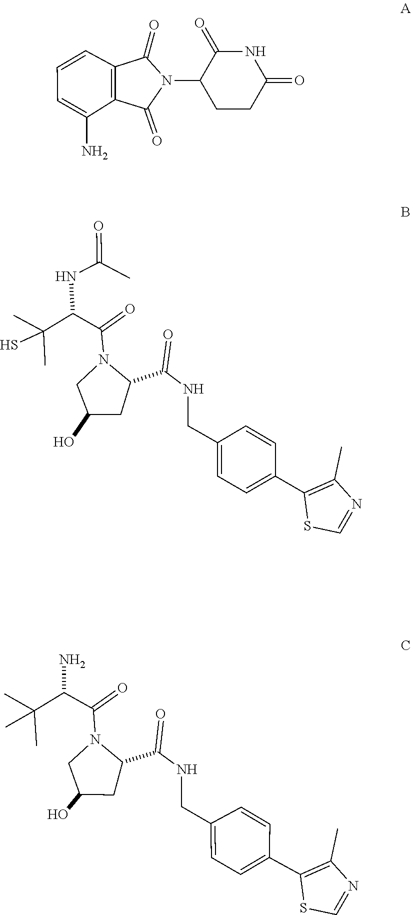

Certain embodiments are directed to pharmaceutical compositions comprising a compound or PROTAC described herein and a suitable pharmaceutical carrier, excipient, or diluent.

Other embodiments are directed to methods of treating cancer, the method comprising administering the composition or a compound described herein to a subject having the cancer. In certain aspects the cancer is selected from brain cancer, salivary cancer, colorectal cancer, myeloma, leukemia, non-small cell lung cancer, colon cancer, cancer of the central nervous system, melanoma, ovarian cancer, renal cancer, prostate cancer, uterine cancer, pancreatic cancer, or breast cancer.

Menin is a protein encoded by the human MEN1 gene. Menin is a putative tumor suppressor associated with multiple endocrine neoplasia type 1 (MEN-1 syndrome). In vitro studies have shown that menin is localized to the nucleus, possesses two functional nuclear localization signals, and inhibits transcriptional activation by JunD. However, the function of this protein is not known. Two variants of a shorter transcript have been identified where alternative splicing affects the coding sequence. Five variants where alternative splicing takes place in the 5′ UTR have also been identified. MAN1 is encoded by the mRNA having a nucleotide sequence that is at least 80% identical to accession number NM-000244, NM_130799, NM_130800, NM_130801, or NM_130802. The MEN1 protein having an amino acid sequence that is at least 80% identical to the amino acids set forth in accession number (protein) NP_000235, NP_570711, NP 570712, NP_570713, or NP 570714.

A “Proteolysis Targeted Chimera” or “PROTAC” compound is a hetero-bi-functional compound containing two ligands connected by a linker unit. A first ligand binds to an E3 ubiquitin ligase protein (for example, VHL) and the other ligand binds to the target protein of interest, thereby bringing the ligase and the target into close proximity.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention.

Throughout this application, the term “about” is used to indicate that a value includes the inherent variation of error for the device, the method being employed to determine the value, or the variation that exists among the study subjects.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

As used herein, the term “IC₅₀” refers to an inhibitory dose that results in 50% of the maximum response obtained.

The term half maximal effective concentration (EC₅₀) refers to the concentration of a drug that presents a response halfway between the baseline and maximum after some specified exposure time.

The terms “inhibiting,” “reducing,” or “prevention,” or any variation of these terms, when used in the claims and/or the specification includes any measurable decrease or complete inhibition to achieve a desired result.

The use of the term “or” in the claims is used to mean “and/or” unless explicitly indicated to refer to alternatives only or the alternatives are mutually exclusive, although the disclosure supports a definition that refers to only alternatives and “and/or.”

As used herein, the term “patient” or “subject” refers to a living mammalian organism, such as a human, monkey, cow, sheep, goat, dogs, cat, mouse, rat, guinea pig, or species thereof. In certain embodiments, the patient or subject is a primate. Non-limiting examples of human subjects are adults, juveniles, infants and fetuses.

The use of the word “a” or “an” when used in conjunction with the term “comprising” in the claims and/or the specification may mean “one,” but it is also consistent with the meaning of “one or more,” “at least one,” and “one or more than one.”

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustrates an example of Menin PROTAC design with incorporation of spacers on solvent-exposed region of piperidine.

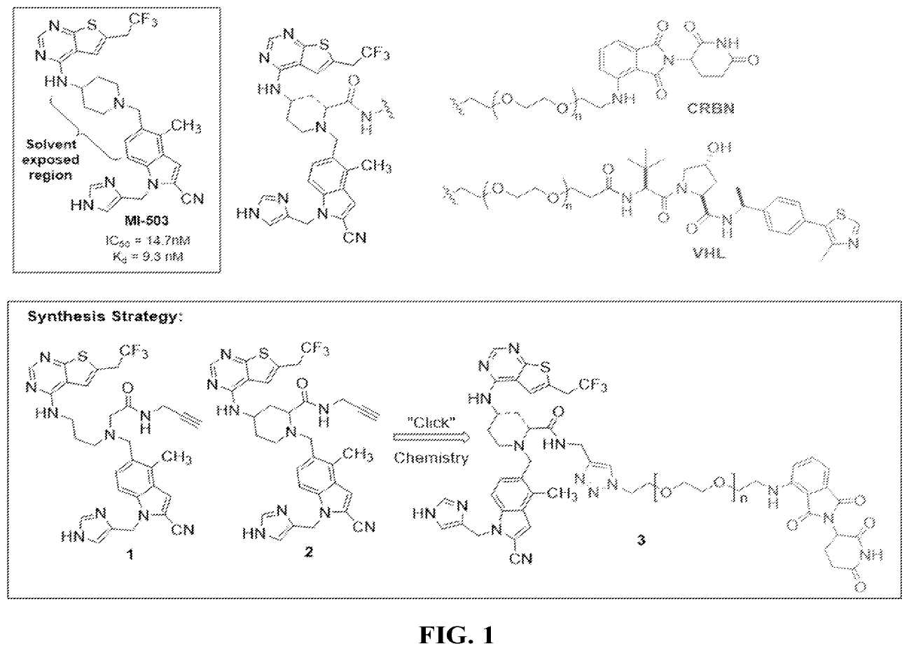

FIG. 2. Illustration of one example of a synthetic scheme for compounds 15 and 16.

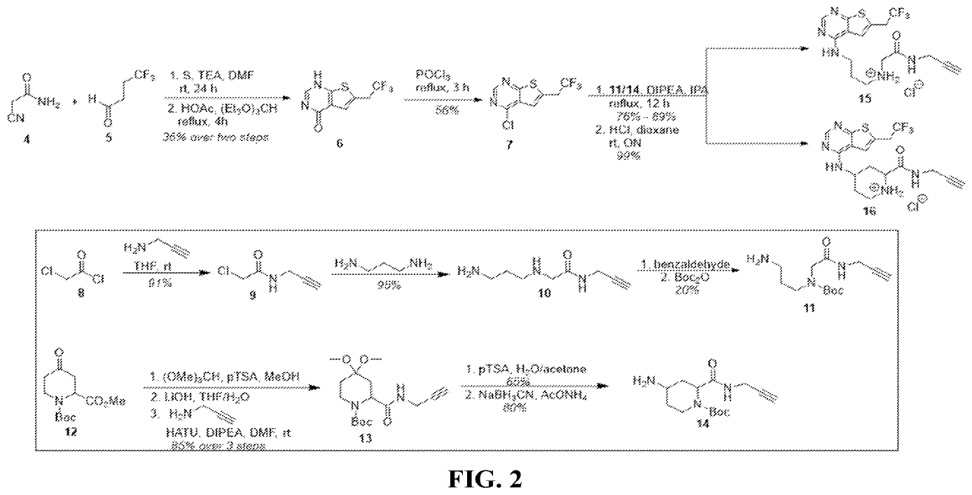

FIG. 3. Illustration of one example of a synthetic scheme for compound 25.

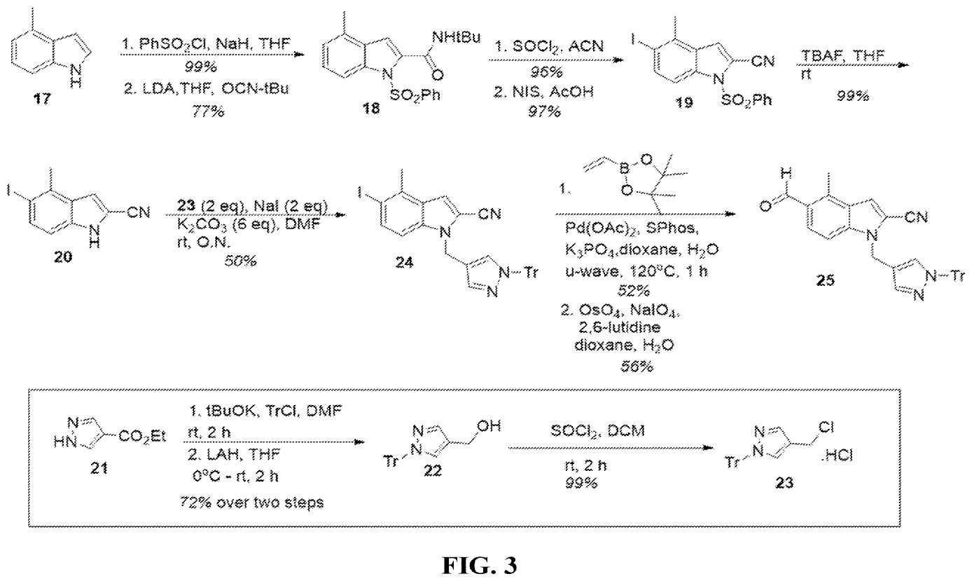

FIG. 4. Illustration of one example of a synthetic scheme for compounds 1 and 2.

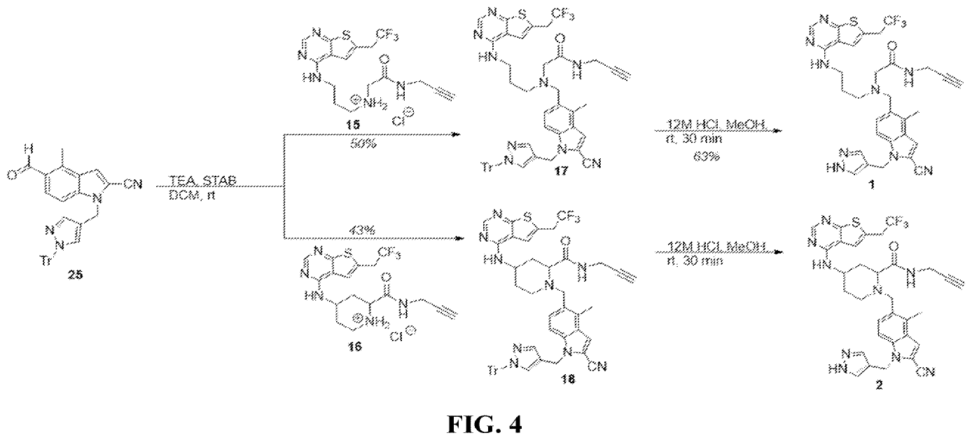

FIG. 5. Illustration of one example of a synthetic scheme for two examples of Menin PROTACS.

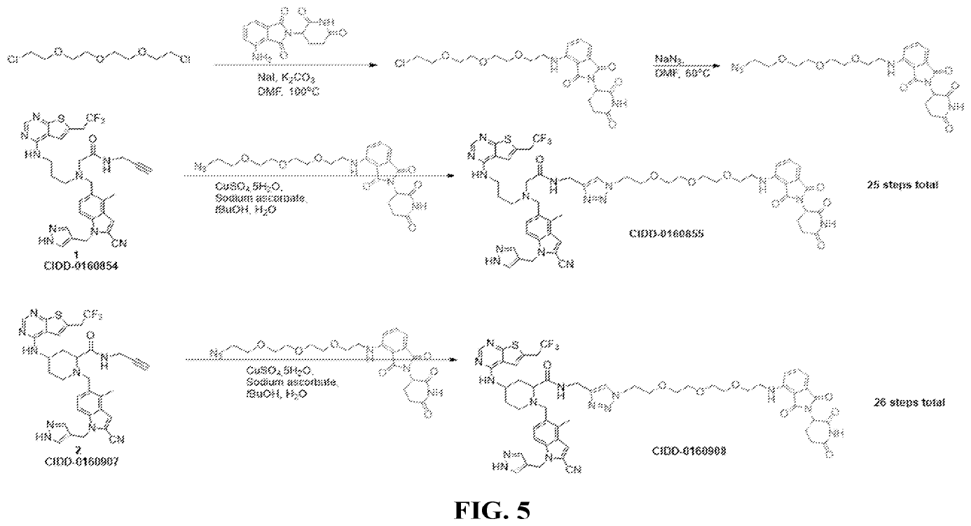

FIG. 6. Illustration of data related to Menin-red-MI-503 binding affinity.

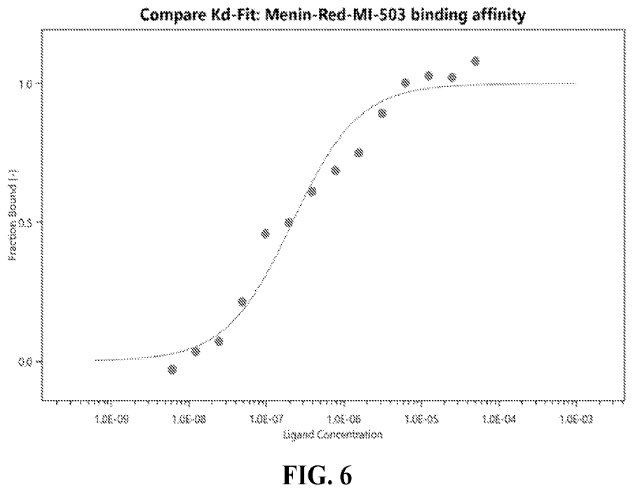

FIG. 7. Illustration of data comparing Menin binding affinity with four PROTAC compounds.

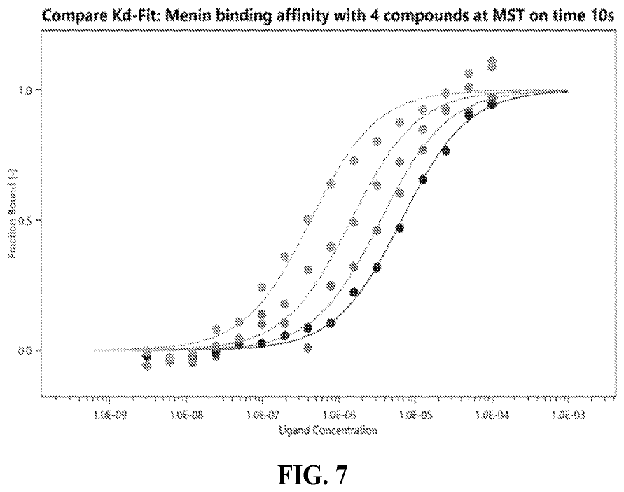

### DESCRIPTION

Menin is a protein encoded by the human MEN1 gene. Menin is a putative tumor suppressor associated with multiple endocrine neoplasia type 1 (MEN-1 syndrome). In vitro studies have shown that Menin is localized to the nucleus, possesses two functional nuclear localization signals, and inhibits transcriptional activation by JunD. However, the function of this protein is not known. Two variants of a shorter transcript have been identified where alternative splicing affects the coding sequence. Five variants where alternative splicing takes place in the 5′ UTR have also been identified. Menin is encoded by an mRNA having a nucleotide sequence that is at least 80% identical to accession number NM 000244, NM_130799, NM_130800, NM_130801, or NM_130802. Menin can have an amino acid sequence that is at least 80% identical to the amino acids set forth in accession number (protein) NP_000235, NP_570711, NP_570712, NP 570713, or NP_570714.

I. Proteolytic-Targeting Chimeric Molecules (PROTACs) that Induce Degradation of Menin Protein

Disclosed herein are proteolytic-targeted chimeric molecules (PROTACs) that induce degradation of Menin protein. In some embodiments, the disclosed molecules may be described as having a having a formula: M-L-E or alternatively E-L-M, wherein Mis a moiety that binds to the Menin protein (e.g., MI-503 or MI-503 derivative)—a Menin binding moiety, L is a linker covalently attaching M and E, and E is a moiety that binds to an E3 ubiquitin ligase—an E3 binding moiety. In certain aspects spacers X₁ couple M and L and X₂ couple E and L.

Certain embodiments described herein are directed to Menin PROTAC's (Proteolysis Targeting Chimeras) as useful therapeutics for the treatment of a variety of cancers and tumors (brain, breast, ovarian, prostate, salivary, etc).

This PROTAC compounds can have the general structure of Formula I or Formula II or a pharmaceutically acceptable salt thereof, wherein:

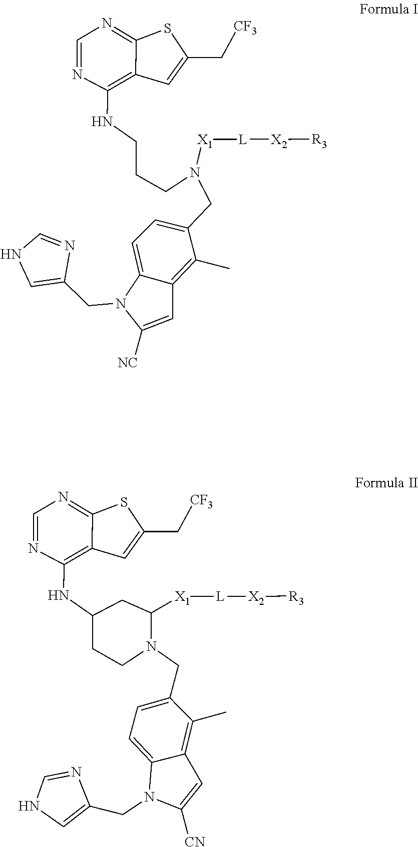

In Formula I, M or the Menin binding moiety is connected to L or spacer X₁ via an amine group.

In Formula II, M or the Menin binding moiety is connected to L or spacer X₁ via a piperidine ring. The bond can be at any position on the piperidine ring.

Certain aspects of the invention are directed to a compound or PROTAC of Formula I or Formula II, wherein:
X₁ and X₂ are independently selected from methylene, nitrogen or oxygen, including —NH—C(O)—, —C(O)—NH—, —C(O)—O—, —O—C(O)—, —O—CH₂—, —NH—CH₂—, —CH₂—NH—, —CH₂—O—, —C(O)—NH—CH₂-triazole-CH₂—, —NH—C(O)—CH₂-triazole-CH₂—, —C(O)—CH₂—, —CH₂—C(O)—, —C(O)—NH—CH₂—, —CH₂—NH—C(O)—, and the like. In certain aspects X₁, X₂, or X₁ and X₂ is substituted. In certain aspects M, E, or M and E can be directly coupled to L without a spacer.
L is a linker selected from alkyl chains (C1-C12), branched alkyl chains (C1-C10), repeating ethylene glycol chains, i.e. (—O—CH₂-CH₂—O—)ₙ, where n is 1-8, ethylene diamines i.e. (—NH—CH₂-CH₂—NH—)ₙ, where n is 1-8, and substituted cyclic amines such as

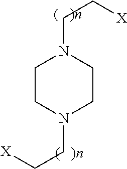

Wherein n=1-6.
R₃ is a suitable small molecule E3-ligase ubiquitin-recruiting ligands such as CRBN ligands such as A below, or VHL ligands such as B and C. The R3 group ligands A, B and C can be optionally substituted to the linkers and X groups on any atom in the structures.

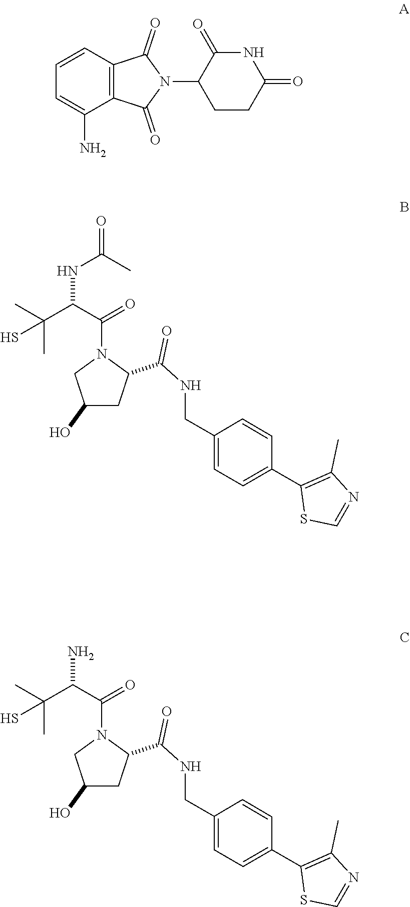 [SMILES: Nc1cccc2c1C(=O)N(C1CCC(=O)NC1=O)C2=O ; CC(=O)N[C@H](C(=O)N1C[C@H](O)C[C@H]1C(=O)NCc1ccc(-c2scnc2C)cc1)C(C)(C)S ; Cc1ncsc1-c1ccc(CNC(=O)[C@@H]2C[C@@H](O)CN2C(=O)[C@@H](N)C(C)(C)S)cc1]

The E3 binding moiety binds to an E3 ubiquitin ligase, for example, as a ligand for the E3 ubiquitin ligase. Ligands for E3 ubiquitin ligases for use in preparing PROTACs are known in the art. (See, e.g., An et al., “Small-molecule PROTACs: An emerging and promising approach for the development of targeted therapy drugs,” EBioMedicine. 2018 October; 36: 553-562; and Gu et al., “PROTACs: An Emerging Targeting Technique for Protein Degradation in Drug Discovery,” Bioessays. 2018 April; 40(4):el700247, the contents of which are incorporated herein by reference in their entireties).

An E3 binding moiety can be a Von Hippel-Lindau (VHL) E3 ubiquitin ligase, cereblon (CRBN) E3 ubiquitin ligase, inhibitor of apoptosis protein (IAP) E3 ubiquitin ligase, and mouse double minute 2 homolog (MDM2) E3 ubiquitin ligase.

An E3 binding moiety can be derived from thalidomide, pomalidomide, lenalidomide, iberdomide, (S,R,S)-AHPC-Me hydrochloride, (S,R,S)-AIPC-Me dihydrochloride, cereblon modulator 1, thalidomide-propargyl, (S,R,S)-AHPC-propargyl, (S,R,S)-AHPC hydrochloride, CC-885, thalidomide-O—COOH, lenalidomide hemihydrate, thalidomide fluoride, thalidomide-OH, lenalidomide-Br, thalidomide D4, lenalidomide hydrochloride, (S,R,S)-AHPC-Me, clAP1 ligand 1, TD-106, E3 ligase Ligand 8, E3 ligase Ligand 9, E3 ligase Ligand 10, E3 ligase Ligand 13, E3 ligase Ligand 14, E3 ligase Ligand 18, BC-1215, VHL ligand 1 (VHL-1), VHL ligand 2 (VHL-2), VHL Ligand 8 (VHL-8), VH032, VH032-cyclopropane-F, VH032 thiol, VH-298, VL-269, VL-285, LCL161, hydroxyproline-based ligands, HIF-1α-derived (R)-hydroxyproline, Nutlin carboxylic acid, (4R,5S)-Nutlin carboxylic acid, (S,R,S)-AHPC-Boc, AR antagonist 1, NV03, (S,R,S)-AHPC TFA, (S,R,S)-AHIPC, β-Naphthoflavone-CH₂—Br, β-Naphthoflavone-CH₂—OH, Bestatin-amido-Me, MV-1-NH-Me, (S,S,S)-AHPC hydrochloride, and clAP1 ligand 2.

The compounds of Formula I or Formula II may be prepared by the methods described below, together with synthetic methods known in the art of organic chemistry, or modifications and derivatizations that are familiar to those of ordinary skill in the art. Preferred methods include, but are not limited to, those described below. During any of the following synthetic sequences, it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; and T. W. Greene and P. G. M. Wuts, Protective Groups in Organic Chemistry, John Wiley & Sons, 1991, which are hereby incorporated by reference.

Compounds of Formula I, Formula II, or their pharmaceutically acceptable salts, can be prepared according to the following reaction Schemes 1-5 below. Isolation and purification of the products is accomplished by standard procedures, which are known to a chemist of ordinary skill.

The following schemes and examples are exemplary of the processes for making compounds of general Formula I and Formula II. It is to be understood, however, that the invention, as fully described herein and as recited in the claims, is not intended to be limited by the details of the following examples.

Scheme 1 provides an example of the synthesis of the intermediate diamines 4 and 7. Referring to Scheme 1, acylation of propargyl amine with chloroacetylchloride 1 produced the desired amide 2. Alkylation with 1,3-propane diamine, followed by selective N-BOC protection provides the desired amine 4. Piperidine 7 was prepare as shown from the N-BOC protected piperidinone 5. Treatment of 5 with pTSA and trimethyl formate, followed by hydrolysis and HATU coupling provides the propargyl amide 6. Deprotection of 6, followed by reductive amination provides the desired amide 7.

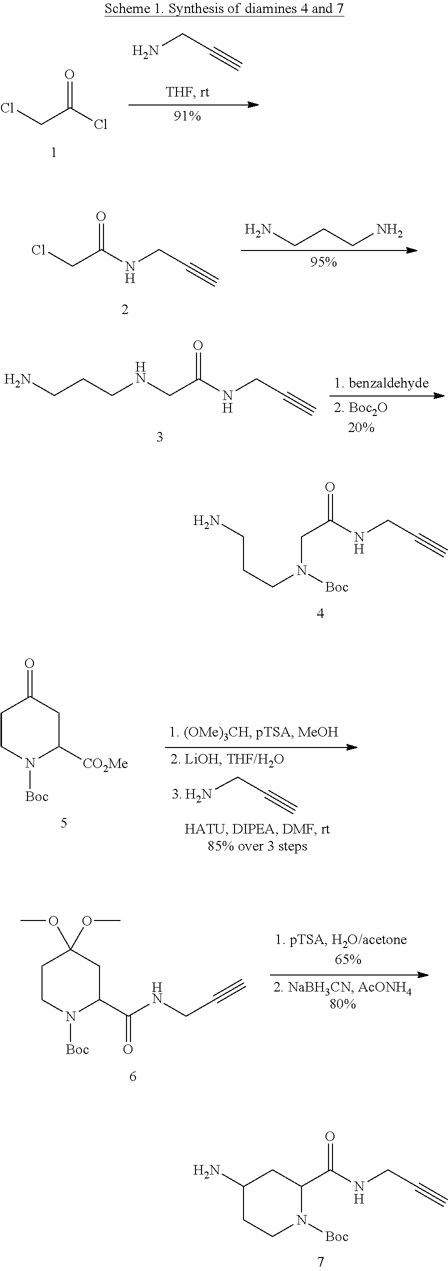

Scheme 2 provide an example of the synthesis of the intermediate propargyl amides 12 and 13. Condensation of 8 and 9 in the presence of sulfur, followed by reaction with triethylorthoformate and acid provides the thio-pyrimidinone 10. Formation of the chloropyrimidine under standard conditions provides compound 11, which is suitable to react with either amine 4 or 7, which provides 12 and 13 after N-BOC deprotection.

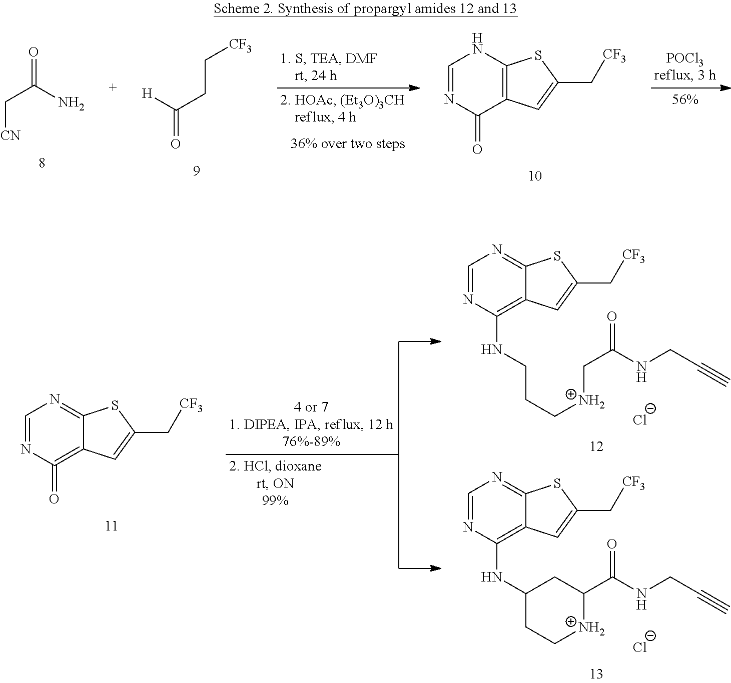

Scheme 3 provides an example of the synthesis of the intermediate indole-aldehyde 19. Treatment of methyl indole 14 with NaH/PhSO₂Cl, followed by LDA/isocyanate provided the desired compound 15. Dehydration and iodoination of 15 provided 16. Deprotection of the benzenesulfonate, followed by alkylation with chloropyrazole 22 provided 18. Finally, Suzuki coupling, followed by oxidative cleavage produced the desired aldehyde 19.

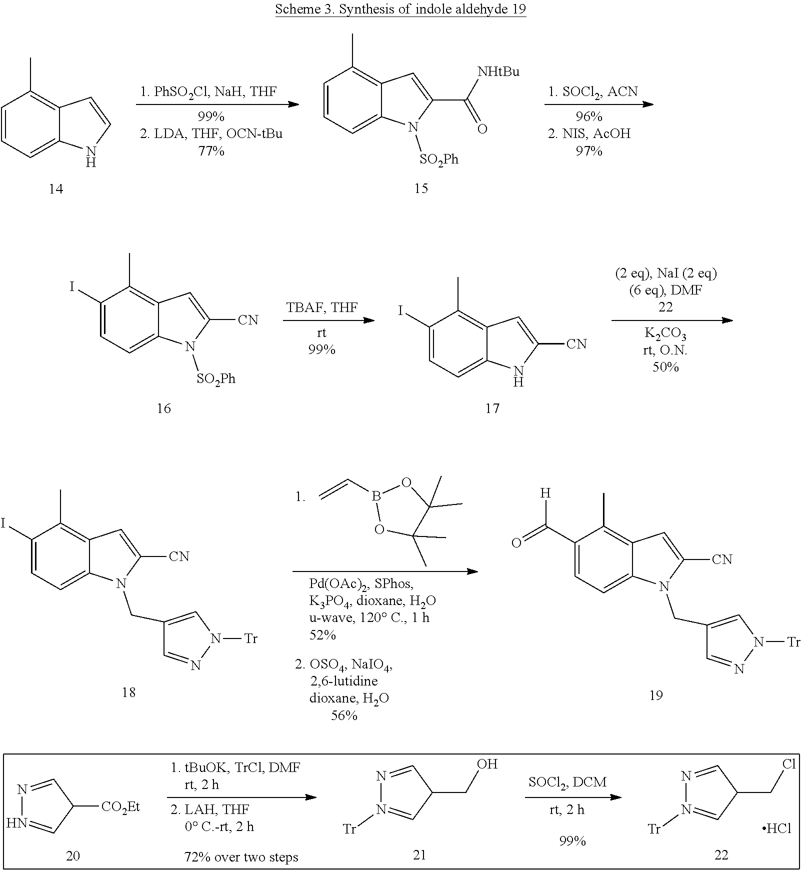

Scheme 4 provides an example of the synthesis of the Menin PROTAC precursors 25 and 26. Reductive amination of aldehyde 19 with either amine 12 or 13, produces the desired amines 23 and 24, respectively. Removal of the trityl group under acidic conditions produces the desired propargyl amide PROTAC precursors, compounds 25 and 26, respectively.

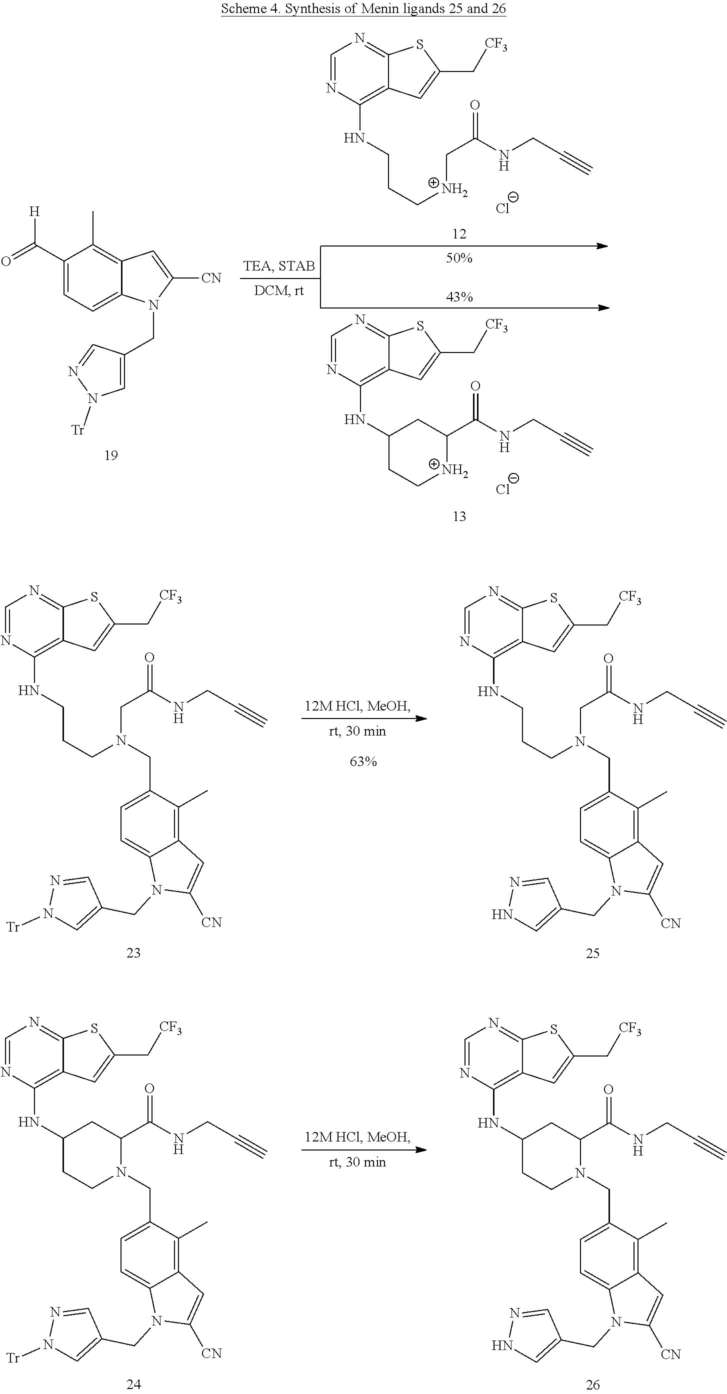

Scheme 5 provides an example of the synthesis of the final Menin PROTAC's, CIDD-0160855 and CIDD-0160908. Treatment of propargyl amines 25 and 26 with the azide 27, under “Click” chemistry conditions (CuSO₄/H₂O), produces the desired triazole PROTAC ligands, CIDD-0160855 and CIDD-0160908, respectively.

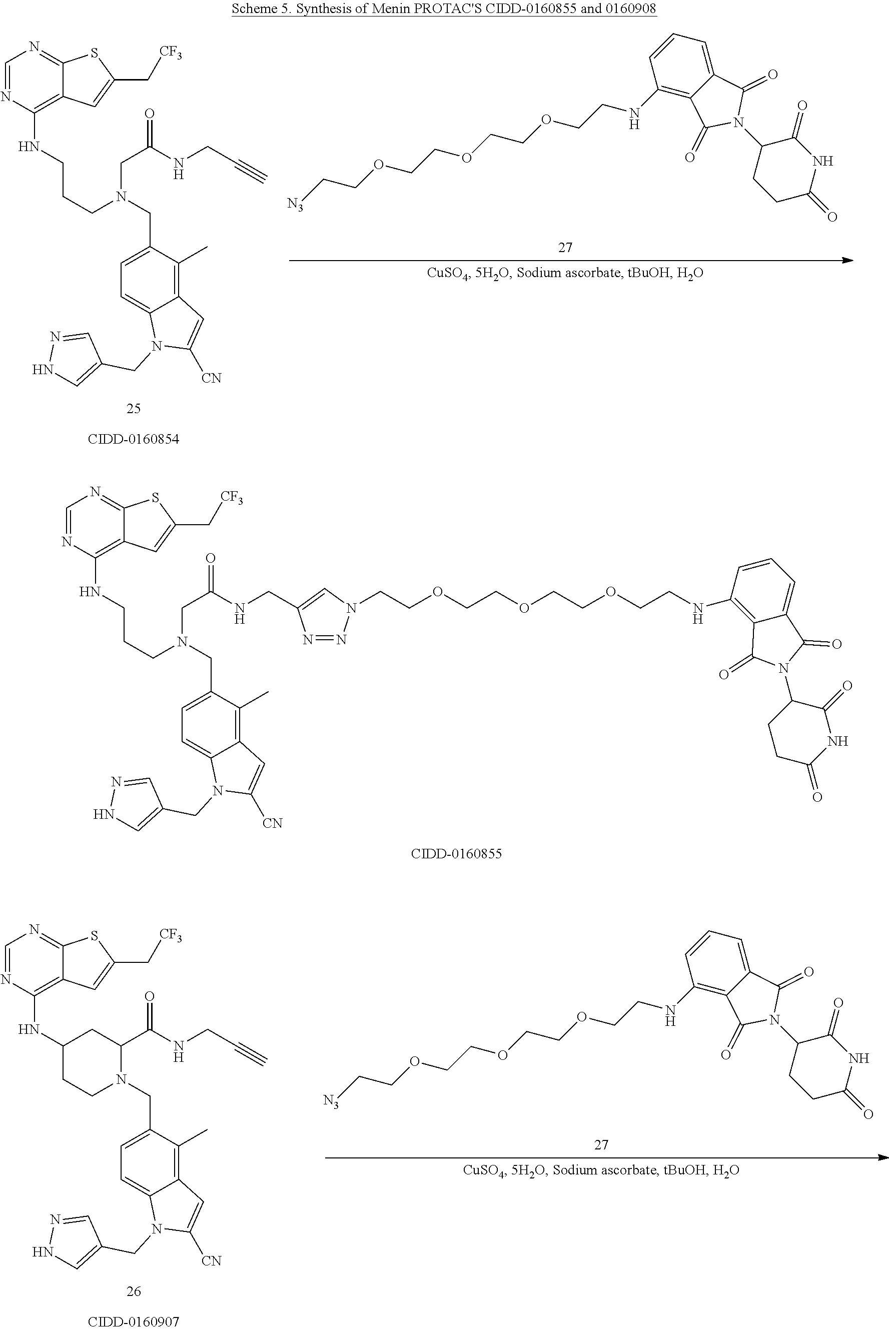

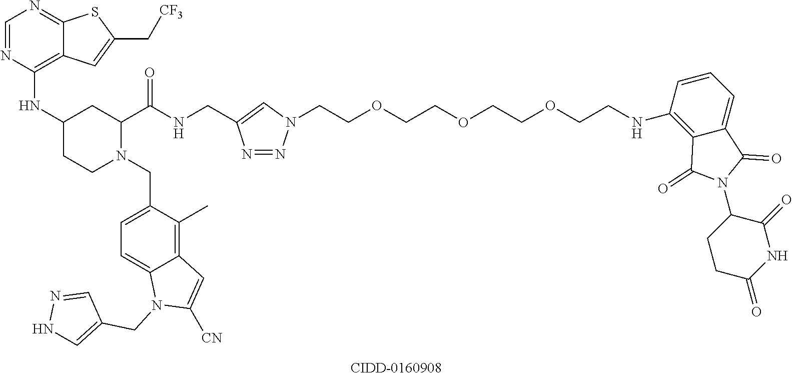 [SMILES: Cc1c(CN2CCC(Nc3ncnc4sc(CC(F)(F)F)cc34)CC2C(=O)NCc2cn(CCOCCOCCOCCNc3cccc4c3C(=O)N(C3CCC(=O)NC3=O)C4=O)nn2)ccc2c1cc(C#N)n2Cc1cn[nH]c1]

Pharmaceutically acceptable salts of the compounds of Formula I or Formula II include the acid or base addition salts thereof. All three reactions are typically carried out in solution. The resulting salt may precipitate out and be collected by filtration or may be recovered by evaporation of the solvent. The degree of ionization in the resulting salt may vary from completely ionized to almost or substantially non-ionized. Suitable non-toxic, acid-addition pharmaceutically acceptable salts include, but are not limited to, the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate and xinofoate salts.

Suitable non-toxic, base-addition pharmaceutically acceptable salts include, but are not limited to, aluminum, arginine, benzathine, calcium, choline, diethylamine, diolamine, glycine, lysine, magnesium, meglumine, olamine, potassium, sodium, tromethamine and zinc salts. For a review on suitable salts, see Handbook of Pharmaceutical Salts: Properties, Selection, and Use by Stahl and Wermuth (Wiley-VCH, 2002).

Included within the scope of the present invention are all stereoisomers, geometric isomers and tautomeric forms of the compounds of Formulas I and Formula II, including compounds exhibiting more than one type of isomerism, and mixtures of one or more thereof.

The present invention includes all pharmaceutically acceptable isotopically-labelled compounds of Formulas I and Formula II wherein one or more atoms are replaced by atoms having the same atomic number, but an atomic mass or mass number different from the atomic mass or mass number which predominates in nature.

Non-limiting examples of compounds of the invention are illustrated in the table below, which include the IUPAC names, structures, CIDD #'s and lot #'s.

| Molecule Name | Structure | Lot ID |
|---|---|---|
| CIDD-0160908 | 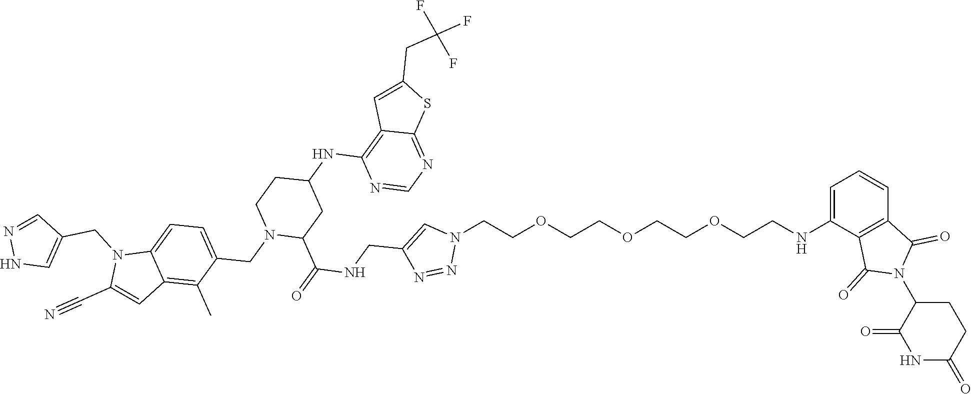 [SMILES: Cc1c(CN2CCC(Nc3ncnc4sc(CC(F)(F)F)cc34)CC2C(=O)NCc2cn(CCOCCOCCOCCNc3cccc4c3C(=O)N(C3CCC(=O)NC3=O)C4=O)nn2)ccc2c1cc(C#N)n2Cc1cn[nH]c1] | SM2019-107-145 |
| CIDD-0160855 | 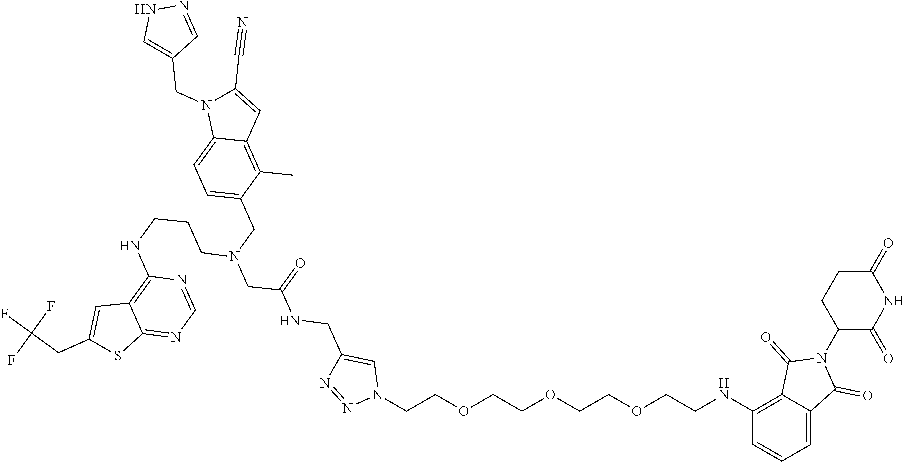 [SMILES: Cc1c(CN(CCCNc2ncnc3sc(CC(F)(F)F)cc23)CC(=O)NCc2cn(CCOCCOCCOCCNc3cccc4c3C(=O)N(C3CCC(=O)NC3=O)C4=O)nn2)ccc2c1cc(C#N)n2Cc1cn[nH]c1] | SM2019-107-99 |

Various chemical definitions related to such compounds are provided as follows.

As used herein, “predominantly one enantiomer” means that the compound contains at least 8500 of one enantiomer, or more preferably at least 9000 of one enantiomer, or even more preferably at least 9500 of one enantiomer, or most preferably at least 990% of one enantiomer. Similarly, the phrase “substantially free from other optical isomers” means that the composition contains at most 500 of another enantiomer or diastereomer, more preferably 2% of another enantiomer or diastereomer, and most preferably 1% of another enantiomer or diastereomer.

As used herein, the term “water soluble” means that the compound dissolves in water at least to the extent of 0.010 mole/liter or is classified as soluble according to literature precedence.

As used herein, the term “nitro” means —NO₂; the term “halo” designates—F, —Cl, —Br or —I; the term “mercapto” means —SH; the term “cyano” means —CN; the term “azido” means —N₃; the term “silyl” means —SiH₃, and the term “hydroxy” means —OH.

The term “alkyl,” by itself or as part of another substituent, means, unless otherwise stated, a linear (i.e. unbranched) or branched carbon chain, which may be fully saturated, mono-or polyunsaturated. An unsaturated alkyl group is one having one or more double bonds or triple bonds. Saturated alkyl groups include those having one or more carbon-carbon double bonds (alkenyl) and those having one or more carbon-carbon triple bonds (alkynyl). The groups, —CH₃ (Me), —CH₂CH₃ (Et), —CH₂CH₂CH₃ (n-Pr), —CH(CH₃)₂(iso-Pr), —CH₂CH₂CH₂CH₃ (n-Bu), —CH(CH₃)CH₂CH₃ (sec-butyl), —CH₂CH(CH₃)₂(iso-butyl), —C(CH₃)₃(tert-butyl), —CH₂C(CH₃)₃ (neo-pentyl), are all non-limiting examples of alkyl groups.

The term “heteroalkyl,” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of O and N. The heteroatom(s) may be placed at any interior position of the heteroalkyl group or at the position at which the alkyl group is attached to the remainder of the molecule. Up to two heteroatoms may be consecutive. The following groups are all non-limiting examples of heteroalkyl groups: trifluoromethyl, —CH₂F, —CH₂ Cl, —CH₂ Br, —CH₂ OH, —CH₂ OCH₃, —CH₂ OCH₂ CF₃, —CH₂OC(O)CH₃, —CH₂ NH₂, —CH₂ NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂ Si(CH₃)₃.

The terms “cycloalkyl” and “heterocyclyl,” by themselves or in combination with other terms, means cyclic versions of “alkyl” and “heteroalkyl”, respectively. Additionally, for heterocyclyl, a heteroatom can occupy the position at which the heterocycle is attached to the remainder of the molecule.

The term “aryl” means a polyunsaturated, aromatic, hydrocarbon substituent. Aryl groups can be monocyclic or polycyclic (e.g., 2 to 3 rings that are fused together or linked covalently). The term “heteroaryl” refers to an aryl group that contains one to four heteroatoms selected from N, O, and S. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl)₂amino, alkylsulfinyl, alkylsulfonyl, arylsulfonyl, unsubstituted cycloalkyl, unsubstituted heterocyclyl, unsubstituted aryl, or unsubstituted heteroaryl. Exemplary optional substituents include, but are not limited to: —OH, oxo (═O), —Cl, —F, Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl).

The term “alkoxy” means a group having the structure —OR′, where R′ is an optionally substituted alkyl or cycloalkyl group. The term “heteroalkoxy” similarly means a group having the structure —OR, where R is a heteroalkyl or heterocyclyl.

The term “amino” means a group having the structure —NR′R″, where R′ and R″ are independently hydrogen or an optionally substituted alkyl, heteroalkyl, cycloalkyl, or heterocyclyl group. The term “amino” includes primary, secondary, and tertiary amines.

The term “oxo” as used herein means an oxygen that is double bonded to a carbon atom.

The term “alkylsulfonyl” as used herein means a moiety having the formula —S(O₂)—R′, where R′ is an alkyl group. R′ may have a specified number of carbons (e.g. “C₁₋₄ alkylsulfonyl”)

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydro fluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base such as methylamine, ethylamine, ethanolamine, lysine, ornithine and the like. Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

It should be recognized that the particular anion or cation forming a part of any salt of this invention is not critical, so long as the salt, as a whole, is pharmacologically acceptable.

Additional examples of pharmaceutically acceptable salts and their methods of preparation and use are presented in Handbook of Pharmaceutical Salts: Properties, Selection and Use (2002), which is incorporated herein by reference.

An “isomer” of a first compound is a separate compound in which each molecule contains the same constituent atoms as the first compound, but where the configuration of those atoms in three dimensions differs. Unless otherwise specified, the compounds described herein are meant to encompass their isomers as well. A “stereoisomer” is an isomer in which the same atoms are bonded to the same other atoms, but where the configuration of those atoms in three dimensions differs. “Enantiomers” are stereoisomers that are mirror images of each other, like left and right hands. “Diastereomers” are stereoisomers that are not enantiomers.

The disclosed PROTACs may be formulated as pharmaceutical compositions. In some embodiments, pharmaceutical compositions as contemplated herein include a PROTAC as disclosed herein, for example, in an effective amount for treating a disease or disorder associated with Menin, and a suitable pharmaceutical carrier, excipient, or diluent.

The disclosed PROTACs and/or pharmaceutical compositions comprising the disclosed PROTACs may be administered to subjects in need thereof, for example, to treat and/or prevent a disease or disorder such as cancer. Suitable cancers treated and/or prevented in the disclosed methods may include, but are not limited to, brain cancer, salivary cancer, colorectal cancer, multiple myeloma, leukemia, non-small cell lung cancer, colon cancer, cancer of the central nervous system, melanoma, ovarian cancer, renal cancer, prostate cancer, pancreatic cancer, and breast cancer.

The disclosed PROTACs may be effective in inhibiting cell proliferation of cancer cells. The disclosed PROTACs may be effective in inhibiting cell proliferation of one or more types of cancer cells including: multiple myeloma cells, such as MM.lS cells; leukemia cells, such as CCRF-CEM, HL-60(TB), MOLT-4, RPMI-8226 and SR; non-small lung cancer cells, such as A549/ATCC, EKVX, HOP-62, HOP-92, NCI-H226, NCI-H23, NCI-H322M, NCI-H460 and NCI-H522; colon cancer cells, such as COLO 205, HCC-2998, HCT-116, HCT-15, HT29, KM12 and SW-620; CNS: SF-268, SF-295, SF-539, SNB-19, SNB-75 and U251; melanoma cancer cells, such as LOX IMVI, MALME-3M, M14, MDA-MB-435, SK-MEL-2, SK-MEL-28, SK-MEL-5, UACC-257 and UACC-62; ovarian cancer cells, such as IGR-OV1, OVCAR-3, OVCAR-4, OVCAR-5, OVCAR-8, NCI/ADR-RES and SK-OV-3; renal cancer cells, such as 786-0, A498, ACHN, CAKI-1, RXF 393, SN12C, TK-10 and UO—31; prostate cancer cells, such as DU-145 and PC-3; pancreatic cancer cells, such as PANC-1, AsPC-1, KP-3, BxPC-3, TCC-PAN2, and MIA PaCa-2. AsPC-1, BxPC-3 and MIA PaCa-2; and breast cancer cells, such as MCF7, MDA-MB-231/ATCC, MDA-MB-468, HS 578T, BT-549 and T-47D.

Cell proliferation and inhibition thereof by the presently disclosed PROTACs may be assessed by cell viability methods disclosed in the art including colorimetric assays that utilize dyes such as MTT, XTT, and MTS to assess cell viability. Preferably, the disclosed PROTACs have an IC₅₀ of less than about 10 μM, 5 μM, 1 μM, 0.5 μM, 0.01 μM, 0.005 μM, 0.001 μM or lower in the selected assay.

The disclosed compounds and molecules (e.g., PROTACs) may be formulated as anti-cancer therapeutics, including hematologic malignancies, brain, salivary, ovarian, breast, lung, pancreas, and prostate malignancies.

The compounds and molecules (e.g., PROTACs) utilized in the methods disclosed herein may be formulated as pharmaceutical compositions that include: (a) a therapeutically effective amount of one or more compounds and molecules as disclosed herein; and (b) one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to about 1000 mg/kg body weight (preferably about 0.5 to about 500 mg/kg body weight, more preferably about 50 to about 100 mg/kg body weight).

The disclosed compounds and molecules and pharmaceutical compositions comprising the disclosed compounds and molecules may be administered in methods of treating a subject in need thereof. For example, in the methods of treatment a subject in need thereof may include a subject having a cell proliferative disease, disorder, or condition such as cancer (e.g., cancers such as brain cancer, salivary cancer, multiple myeloma, leukemia, non-small cell lung cancer, colon cancer, cancer of the central nervous system, melanoma, ovarian cancer, renal cancer, prostate cancer, pancreatic cancer, and breast cancer).

In some embodiments of the disclosed treatment methods, the subject may be administered a dose of a compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds and molecules may include doses falling within dose ranges having as end-points any of these disclosed doses (e.g., 2.5 mg-200 mg).

In some embodiments, a minimal dose level of a compound for achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. In some embodiments, a maximal dose level of a compound for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. Minimal and/or maximal dose levels of the compounds and molecules for achieving therapy in the disclosed methods of treatment may include dose levels falling within ranges having as end-points any of these disclosed dose levels (e.g., 500-2000 ng/kg body weight of the subject).

The compounds and molecules utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The compounds and molecules utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds and molecules utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds and molecules such as phenol, or quaternary compounds and molecules such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

The compounds and molecules utilized in the methods disclosed herein may be formulated as a pharmaceutical composition for delivery via any suitable route. For example, the pharmaceutical composition may be administered via oral, intravenous, intramuscular, subcutaneous, topical, and pulmonary route. Examples of pharmaceutical compositions for oral administration include capsules, syrups, concentrates, powders and granules. In some embodiments, the compounds and molecules are formulated as a composition for administration orally (e.g., in a solvent such as 5% DMSO in oil such as vegetable oil).

The compounds and molecules utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds and molecules may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are preferably applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

It is contemplated that any embodiment discussed in this specification can be implemented with respect to any method or composition of the invention, and vice versa. Furthermore, compositions of the invention can be used to achieve methods of the invention.

1. A compound having the structure:
M-L-E, E-L-M, or a pharmaceutically acceptable salt thereof, wherein
M is a Menin protein binding moiety;
E is an E3 ubiquitin ligase protein binding ligand; and
L is a linker group.

2. The compound of claim 1, wherein the compound has a structure of Formula I or Formula II: 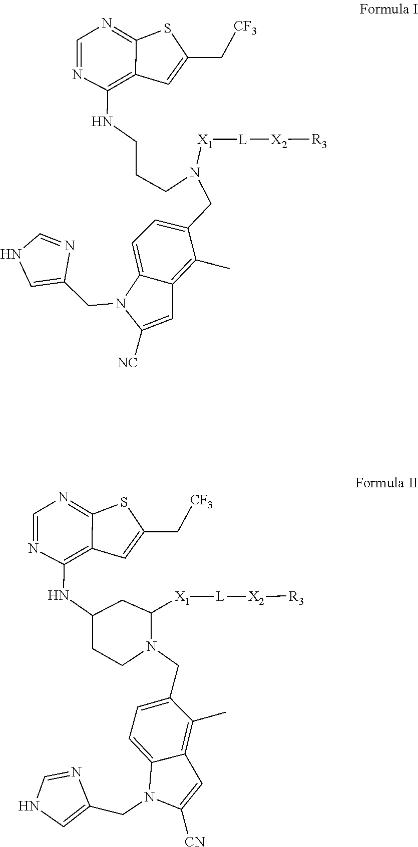
wherein X₁ and X₂ are independently selected from an optionally substituted methylene, nitrogen or oxygen, including —NH—C(O)—, —C(O)—NH—, —C(O)—O—, —O—C(O)—, —O—CH₂—, —NH—CH₂—, —CH₂—NH—, —CH₂—O—, —C(O)—NH—CH₂-triazole-CH₂—, —NH—C(O)—CH₂-triazole-CH₂—, —C(O)—CH₂—, —CH₂—C(O)—, —C(O)—NH—CH₂—, or —CH₂—NH—C(O)—;
L is a linker; and
R₃ is an E3 binding moiety.

3. The compound of claim 1, wherein L is selected from alkyl chains (C1-C12), branched alkyl chains (C1-C10), repeating ethylene glycol chains, i.e. (—O—CH₂—CH₂—O—)ₙ, where n is 1-8, ethylene diamines i.e. (—NH—CH₂—CH₂—NH—)ₙ, where n is 1-8, and substituted cyclic amines.

4. The compound of claim 3, wherein the substituted cyclic amin has a structure of 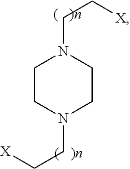
wherein n=1-6.

5. The compound of claim 1, wherein E3 binding moiety is selected from a moiety that binds Von Hippel-Lindau (VHL) E3 ubiquitin ligase, moiety that binds cereblon (CRBN) E3 ubiquitin ligase, moiety that binds inhibitor of apoptosis protein (IAP) E3 ubiquitin ligase, or moiety that binds mouse double minute 2 homolog (MDM2) E3 ubiquitin ligase.

6. The compound of claim 1, wherein the E3 binding moiety is selected from:

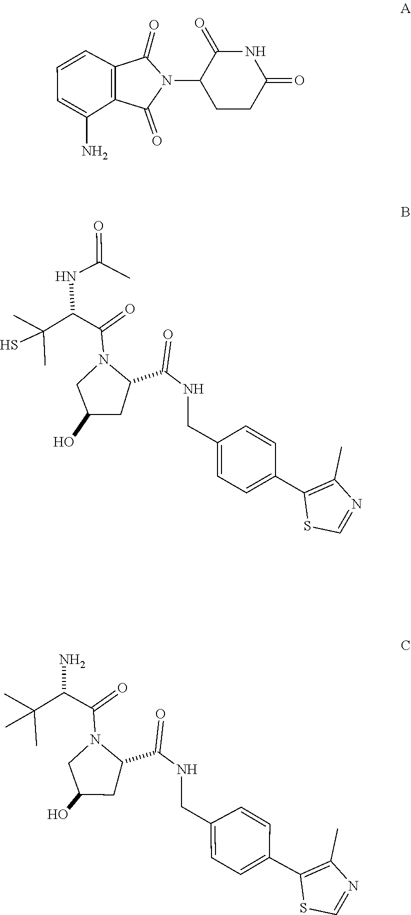

7. A pharmaceutical composition comprising the compound of claim 1 and a suitable pharmaceutical carrier, excipient, or diluent.

8. A method of treating cancer, the method comprising administering the composition of claim 1 to a subject having the cancer.

9. The method of claim 8, wherein the cancer is selected from brain cancer, salivary cancer, colorectal cancer, myeloma, leukemia, non-small cell lung cancer, colon cancer, cancer of the central nervous system, melanoma, ovarian cancer, renal cancer, prostate cancer, uterine cancer, pancreatic cancer, or breast cancer.
